(19) United States
(12) Patent Application Publication
(10) Pub. No.: US 2026/0216700 A1
(43) Pub. Date: Jul. 30, 2026

(54) CHROMIUM ALUMINA CATALYSTS FOR PARAFFIN DEHYDROGENATION

(71) Applicant: SABIC GLOBAL TECHNOLOGIES B.V., Bergen op Zoom (NL)

(72) Inventors: Biju Maippan DEVASSY, Bangalore (IN); Vinod Sankaran NAIR, Bangalore (IN); Velayutham SARAVANAN, Hosur (IN); Nigit Jose MELEPPURAM, Bangalore (IN)

(21) Appl. No.: 19/141,481

(22) PCT Filed: Dec. 18, 2023
(86) PCT No.: PCT/EP2023/086285

§ 371 Date: Jun. 20, 2025

Related U.S. Application Data
(60) Provisional application No. 63/476,642, filed on Dec. 22, 2022.

(51) Int. Cl. B01J 23/26; B01J 37/02; B01J 37/12; C07C 5/333

(52) U.S. Cl. CPC B01J 23/26; B01J 37/0201; B01J 37/12; C07C 5/3332; C07C 2521/04; C07C 2523/26

(57) ABSTRACT

Methods of preparing and using alkane dehydrogenation catalysts are provided. The method for making an alkane dehydrogenation catalyst may include providing a chromium(III) oxide-containing alumina support derived from a plurality of transition aluminas, contacting the alumina support with a water-soluble trivalent chromium oxide source and an alkali metal oxide source to provide an impregnated alumina support, and drying and calcining the impregnated alumina support to produce an alkane dehydrogenation catalyst. The catalyst can contain about 60 wt. % to about 99 wt. % of alumina, about 1 wt. % to about 40 wt. % of trivalent chromium oxide, and about 0.1 wt. % to about 5 wt. % of the alkali metal oxide.

### CROSS-REFERENCE TO RELATED APPLICATIONS

This application claims the benefit of priority to U.S. Provisional Patent Application No. 63/476,642, filed Dec. 22, 2022, the entire contents of which are hereby incorporated by reference in their entirety.

### TECHNICAL FIELD

The present disclosure generally relates to methods for preparing and using an alkane dehydrogenation catalyst. More specifically, the present disclosure relates to, among other embodiments, methods for the use and preparation of chromia-alumina dehydrogenation catalysts for the dehydrogenation of paraffins.

### BACKGROUND

Alkane dehydrogenation is a recognized process for the production of a variety of useful hydrocarbon products, such as isobutylene for conversion to MTBE, as well as isooctane and alkylates to supplement and enrich gasolines. There are several current catalytic processes useful for the catalytic dehydrogenation of light alkanes, including the Süd-Chemie CATOFIN® process, UOP's Oleflex® process, Phillips' Star™ process, and the Snamprogetti-Yarsintee process. The catalysts that are used in these processes are manufactured from two different groups of materials. The Süd-Chemie CATOFIN® process and the Snamprogetti-Yarsintee process utilize chromia-alumina catalysts. In contrast, the catalysts for the UOP and Phillips processes include supported precious metal platinum as catalysts.

Chromia-alumina dehydrogenation catalyst technology has been in use for many decades. One of the important requirements in the production of pelleted/extruded catalysts for use in fixed-bed catalytic operations is that the catalyst be of sufficient strength to support its own weight as well as the process-stream turbulence and similar disturbances ordinarily encountered in fixed-bed processes. Failure to satisfy these requirements is a frequent source of difficulty. Additionally, the gradual loss of mechanical strength during use is a common occurrence necessitating the discarding of catalyst which in other respects would continue to be entirely satisfactory. Accordingly, the Applicant has recognized that there exists a need for a simple, cost effective method for making a chromia-alumina dehydrogenation catalyst, and which exhibits good activity, improved stability, and suitable mechanical strength, such as crush strength, for the dehydrogenation of paraffins.

### SUMMARY

To address shortcomings in the art, the Applicant has developed methods for the preparation and use of alkane dehydrogenation catalyst. Embodiments of methods for making an alkane dehydrogenation catalyst include the steps of providing a chromium(III) oxide-containing alumina support containing a plurality of transition aluminas derived from a mixture containing about 60 weight percent (wt. %) to about 99 wt. % of crystalline aluminium trihydroxide and about 1 wt. % to about 40 wt. % of crystalline aluminium oxide-hydroxide or gelatinous aluminium hydroxide or both, or preferably from about 60 wt. % to about 97 wt. % of crystalline aluminum trihydroxide and from about 3 wt. % to about 40 wt. % of crystalline aluminum oxide-hydroxide or gelatinous aluminum hydroxide, or combinations thereof; and more preferably from about 90 wt. % to about 99 wt. % of crystalline aluminum trihydroxide and from about 10 wt. % to about 40 wt. % of crystalline aluminum oxide-hydroxide or gelatinous aluminum hydroxide, or combinations thereof. The method further includes the step of contacting the chromium(III) oxide-containing alumina support with a water-soluble trivalent chromium oxide source and an alkali metal oxide source to provide an impregnated chromium(III) oxide-containing alumina support, and drying and calcining the impregnated chromium(III) oxide-containing alumina support to produce an alkane dehydrogenation catalyst containing about 60 wt. % to about 90 wt. % of alumina, about 10 wt. % to about 40 wt. % of trivalent chromium oxide, and about 0.1 wt. % to about 5 wt. % of the alkali metal oxide. In certain embodiments, the alkane dehydrogenation catalyst contains about 70 wt. % to about 90 wt. % of alumina, about 10 wt. % to about 30 wt. % of trivalent chromium oxide, and about 0.1 wt. % to about 5 wt. % of the alkali metal oxide. In certain embodiments, the crystalline aluminium trihydroxide contains one or more of bayerite and nordstrandite. The crystalline aluminium oxide-hydroxides can contain boehmite. The gelatinous aluminium hydroxides can contain one or more of amorphous aluminium hydroxide and pseudoboehmite. In certain embodiments, the impregnated chromium(III) oxide-containing alumina support is calcined at a temperature ranging from about 700 degrees Celsius (° C.) to 800° C.

Embodiments disclosed herein also include methods for using an alkane dehydrogenation catalyst. One such method for dehydrogenation of an alkane includes the steps of loading a reactor with a dehydrogenation catalyst prepared as disclosed herein and supplying a feed containing an alkane through the reactor at a temperature sufficient to dehydrogenate the alkane and produce an olefin. In certain embodiments, the temperature sufficient to dehydrogenate the alkane ranges from 400° C. to 800° C.

Still other aspects and advantages of these exemplary embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description. Furthermore, it is to be understood that the features of the various embodiments described herein are not mutually exclusive and may exist in various combinations and permutations.

### DETAILED DESCRIPTION

The present disclosure describes various embodiments related to methods for preparing and using an alkane dehydrogenation catalyst. Further embodiments may be described and disclosed.

In the following description, numerous details are set forth in order to provide a thorough understanding of the various embodiments. In other instances, well-known processes, devices, and systems may not have been described in particular detail to not unnecessarily obscure the various embodiments. Additionally, illustrations of the various embodiments may omit certain features or details to not obscure the various embodiments.

The description may use the phrases “in some embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “about” refers to an acceptable error for a particular value as determined by one of ordinary skill in the art using measurements in accordance with the referenced standards for the experiments. In one non-limiting embodiment, when the term “about” is used with a particular value, then “about” refers to a range extending to +10% of the specified value, alternatively ±5% of the specified value, or alternatively ±1% of the specified value, or alternatively ±0.5% of the specified value. In embodiments, “about” refers to the specified value.

The use of the words “a” or “an” when used with any of the terms “comprising,” “including,” “containing,” or “having,” in the claims or the specification may mean “one,” but it is also consistent with the meaning of “one or more,” “at least one,” and “one or more than one.”

The terms “wt. %”, “vol. %”, or “mol. %” refer to a weight, volume, or molar percentage of a component, respectively, based on the total weight, the total volume of material, or total moles, that includes the component. In a non-limiting example, 10 grams of component in 100 grams of the material is 10 wt. % of component.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The terms “a mixture thereof” and “a combination thereof” or any variation of these terms, when used in the claims and/or the specification in conjunction with a list of components, means any combination of two or more of the listed components, including such combinations in which one or more of the other listed components are absent therefrom.

Disclosed here are methods for preparing and using an alkane dehydrogenation catalyst. The dehydrogenation catalysts prepared according to the presently disclosed methods have good activity as well as improved stability and mechanical strength.

Embodiments for a method for making an alkane dehydrogenation catalyst include the steps of providing a chromium(III) oxide-containing alumina support containing a plurality of transition aluminas derived from a mixture containing about 60 wt. % to about 99 wt. % of crystalline aluminium trihydroxide and about 1 wt. % to about 40 wt. % of crystalline aluminium oxide-hydroxide or gelatinous aluminium hydroxide or both, or preferably containing about 60 wt. % to about 97 wt. % of crystalline aluminium trihydroxide and about 3 wt. % to about 40 wt. % of crystalline aluminium oxide-hydroxide or gelatinous aluminium hydroxide or both, or more preferably about 90 wt. % to about 99 wt. % of crystalline aluminium trihydroxide and about 1 wt. % to about 10 wt. % of crystalline aluminium oxide-hydroxide or gelatinous aluminium hydroxide or both. The present disclosure is directed to the use of at least two types of aluminum hydroxides along with a chromium(III) oxide source, optionally an alkali metal oxide source, and an aqueous acid to form the chromium(III) oxide-containing alumina support. The transition alumina formed from the mixture of aluminum hydroxides is texpected to increase crush strength without affecting the catalyst performance up to a particular combination of aluminum hydroxides.

The method further includes the step of contacting the chromium(III) oxide-containing alumina support with a water-soluble trivalent chromium oxide source and an alkali metal oxide source to provide an impregnated chromium(III) oxide-containing alumina support, and drying and calcining the impregnated chromium(III) oxide-containing alumina support to produce an alkane dehydrogenation catalyst containing about 60 wt. % to about 90 wt. % of alumina, about 10 wt. % to about 40 wt. % of trivalent chromium oxide, and about 0.1 wt. % to about 5 wt. % of the alkali metal oxide.

The chromium(III) oxide-containing alumina support contains a plurality of transition aluminas derived from a mixture containing crystalline aluminium trihydroxide and one or more of crystalline aluminium oxide-hydroxide and gelatinous aluminium hydroxide.

“Transition aluminas” are one or more aluminas other than alpha-alumina, which are capable of being at least partially converted to alpha-alumina under thermal treatment at 900° C. or greater. Transition aluminas include, but are not limited to gamma-alumina, delta-alumina, eta-alumina, kappa-alumina, chi-alumina, rho-alumina, and theta-alumina. In certain embodiments, the transition alumina support is substantially an eta-alumina. An alumina support that contains substantially eta-alumina refers to an alumina support containing from about 60 wt. % to about 99 wt. % of eta-alumina and all ranges and values therebetween, such as from 60 wt. % to 90 wt. %, 65 wt. % to about 90 wt. %, about 75 wt. % to about 90 wt. %, about 80 wt. % to about 90 wt. %, 90 to 99 wt. % about 80 wt. %, about 85 wt. %, or about 85 wt. %, or about 87 wt. %, or about 90 wt. %.

In certain embodiments, the method for making a chromium(III) oxide-containing alumina support may include mixing a plurality of transition aluminas, a water insoluble chromium(III) oxide source, and optionally an alkali metal oxide source with an aqueous metal free acid solution to form a moldable mixture. The method may further include extruding the moldable mixture to form extrudates and drying and calcining the extrudates to produce a chromium(III) oxide-containing alumina support containing a plurality of transition aluminas. The alkali metal oxide sources can contain a sodium oxide, lithium oxide, potassium oxide, or cesium oxide source. The various components can be mixed by a variety of methods, both manual and mechanical, to provide the moldable mixture. For example, in certain embodiments, the moldable mixture may be mixed by a batch mixer. The raw materials used for the catalyst preparation can be mixed well in a high shear mixer followed by mixing with a metal free aqueous acid solution until a rather stiff dough/granules are obtained. This dough/granules can be extruded and/or formed into any suitable shape including cylinders, cubes, stars, tri-lobes, quadra-lobes, pellets, pills, or spheres by suitable mechanical means. In one embodiment, mixing is conducted in a high intensity environment, such as that supplied by a B&P Littleford Mixer available from B&P Littleford, 1000 Hess Avenue, Saginaw, MI 48601.

In another embodiment, mixing is conducted using an Eirich Intensive Mixer, such as that supplied by Maschinenfabrik GustavEirich Gmbh & Co KG, Hardheim, Germany. Mixing is conducted for a time sufficient to result in a uniform mixture. In other embodiments, other batch or continuous processes can be used to create the moldable mixture. Components may be added serially or together in any convenient order, as would be apparent to the person of ordinary skill in the art. In certain embodiments of the method for making an alkane dehydrogenation catalyst, the extrudates may be dried to remove water by heating at a temperature of 50° C. to 200° C., 100° C. to 140° C., 110° C. to 120° C., or 50° C., 55° C., 60° C., 65° C., 70° C., 75° C., 80° C., 85° C., 90° C., 95° C., 100° C., 105° C., 110° C., 115° C., 120° C., 125, ° C., 130° C., 135° C., 140° C., 145° C., 150° C., or any range or value therebetween.

In certain embodiments of the method for making an alkane dehydrogenation catalyst, the extrudates may be calcined at a temperature ranging from about 500° C. to about 900° C. In certain embodiments, the presently described alkane dehydrogenation catalyst may be in the form of extrudates having a diameter of from about 1 to about 4 millimeters, or from about 2 to about 3.5 millimeters, and a length of from about 2 to about 10 millimeters, or from about 3 to about 9 millimeters. In certain embodiments, the aqueous metal free acid solution includes acids, such as nitric acid. The moldable mixture includes an aqueous non-metal acid. The non-metal acid herein refers to an acid in which the molecular structure of the acid does not involve a metal atom. In certain embodiments of the process, as otherwise described herein, the non-metal acid may be nitric acid. In other embodiments of the process, as otherwise described herein, the non-metal acid may be an organic acid, such as formic acid or acetic acid. In still other embodiments of the process, the non-metal acid may be a combination of nitric acid and an organic acid such as formic acid or acetic acid. In certain embodiments, the use of an organic acid can be beneficial in that it can reduce the nitrogen oxides concentration during heat treatment. However, it can also make the peptization of aluminum hydroxides less efficient. The person of ordinary skill in the art will determine the appropriate amounts and types of acids to use to provide a desired moldable material.

In certain embodiments, the amount of crystalline aluminium trihydroxide ranges from about 60 wt. % to about 99 wt. %, from about 60 wt. % to about 97 wt. %, and including values between these values, such as from about 62 wt. % to about 95 wt. %, from about 65 wt. % to about 97 wt. %, from about 67 wt. % to about 97 wt. %, from about 70 wt. % to about 97 wt. %, from about 72 wt. % to about 97 wt. %, from about 74 wt. % to about 97 wt. %, from about 75 wt. % to about 97 wt. %, from about 77 wt. % to about 97 wt. %, from about 60 wt. % to about 95 wt. %, from about 60 wt. % to about 93 wt. %, or from about 60 wt. % to about 90 wt. %, or from about 85 wt. % to about 97 wt. %, or from about 80 wt. % to about 97%, from about 90 to about 97 wt. % or from about 90 wt. % to 99 wt. %. In certain embodiments, the crystalline aluminium trihydroxide contains one or more of bayerite and nordstrandite.

Embodiments also include crystalline aluminium oxide-hydroxide or gelatinous aluminium hydroxide or both ranging from about 1 wt. % to about 40 wt. %, and including values between these values, such as from about 3 wt. % to about 40 wt. %, 5 wt. % to about 40 wt. %, from about 8 wt. % to about 40 wt. %, from about 10 wt. % to about 40 wt. %, from about 15 wt. % to about 40 wt. %, from about 3 wt. % to about 35 wt. %, from about 3 wt. % to about 30 wt. %, from about 5 wt. % to about 30 wt. %, from about 3 wt. % to about 25 wt. %, from about 5 wt. % to about 25 wt. %, or from about 3 wt. % to about 20 wt. %, or from about 3 wt. % to about 15 wt. %, from about 3 wt. % to about 10 wt. % or from about 1 wt. % to about 10 wt. %. The crystalline aluminium oxide-hydroxides can contain boehmite. The gelatinous aluminium hydroxides can contain one or more of amorphous aluminium hydroxide and pseudoboehmite.

The impregnation solution that is brought into contact with the chromium(III) oxide-containing alumina support contains a water-soluble trivalent chromium oxide source and an alkali metal oxide source. The chromium oxide source can include chromium nitrate or a chromium acetate. In various embodiments of the impregnation methods, the water-soluble trivalent chromium oxide source is present in the aqueous solution in an amount ranging from about 20 wt. % to about 50 wt. %, e.g., about 22 wt. % to about 50 wt. %, or about 25 wt. % to about 50 wt. %, or about 27 wt. % to about 50 wt. %, or about 30 wt. % to about 50 wt. %, or about 20 wt. % to about 48 wt. %, or about 20 wt. % to about 45 wt. %, or the amount is about 25 wt. %, or about 30 wt. %, or about 35 wt. %, or about 37.5 wt. %, or about 40 wt. %, or about 45 wt. %.

The alkali metal oxide source can be an alkali metal salt. In certain embodiments, the alkali metal salt is a sodium salt or a lithium salt or a potassium salt. In certain embodiments, the alkali metal salt source can be a hydroxide, a carbonate, or an acetate. The alkali metal salt is present in the impregnation solution in a concentration sufficient to provide the final dehydrogenation catalyst with the alkali metal oxide in an amount ranging from about 0.1 wt. % to about 5 wt. %. For example, the alkali metal salt can be provided in the impregnation solution to yield the final dehydrogenation catalyst with the alkali metal oxide in an amount ranging from about 0.3 wt. % to about 5 wt. %, about 0.5 wt. % to about 5 wt. %, or about 1 wt. % to about 5 wt. %, or about 0.1 wt. % to about 4.5 wt. %, or about 0.1 wt. % to about 4 wt. %, or about 0.25 wt. % to about 4.75 wt. %, or about 0.2 wt. % to about 3.5 wt. %, or about 0.25 wt. % to about 3.5 wt. %, or about 0.5 wt. % to about 2.5 wt. %, or about 1 wt. %, or about 2 wt. %, or about 3 wt. %, or about 4 wt. %, or about 4.5 wt. %, calculated as alkali metal oxide on a calcined basis.

5 In certain embodiments, the impregnated chromium(III) oxide-containing alumina support may be dried to remove water by heating at a temperature of 50° C. to 200° C., 100° C. to 140° C., 110° C. to 120° C., or 50° C., 55° C., 60° C., 65° C., 70° C., 75° C., 80° C., 85° C., 90° C., 95° C., 100° C., 105° C., 110° C., 115° C., 120° C., 125, ° C., 130° C., 135° C., 140° C., 145° C., 150° C., or any range or value therebetween. In certain embodiments, the impregnated chromium(III) oxide-containing alumina support is calcined at a temperature ranging from about 700° C. to 800° C.

Embodiments disclosed here also include methods for using an alkane dehydrogenation catalyst. One such method for dehydrogenation of an alkane includes the steps of loading a reactor with a dehydrogenation catalyst prepared as disclosed herein and supplying a feed containing an alkane through the reactor at a temperature sufficient to dehydrogenate the alkane and produce an olefin. In certain embodiments, the method may further include separating a dehydrogenated product from unreacted alkanes. In certain embodiments, the temperature sufficient to dehydrogenate the alkane ranges from 400° C. to 800° C.

The feed can include C2 to C20 linear, iso, and cyclo-alkanes, which are substantially saturated compounds containing hydrogen and carbon. The feed encompasses a range of cyclic and linear alkanes, such as ethane, propane, n-butane, isobutane, n-pentane, isopentane, n-hexane, 2-methylpentane, 3-methylpentane, 2,2-dimethylbutane, n-heptane, 2-methylhexane, 2,2,3-trimethylbutane, cyclopentane, cyclohexane, methylcyclopentane, ethylcyclopentane, n-propylcyclopentane and 1,3-dimethylcyclohexane. For example, propane can be used as an alkane-containing feed, which can be dehydrogenated to produce propylene, and isobutane can be used as an alkane-containing feed, which can be dehydrogenated to produce isobutylene. In certain aspects, substantially all of the hydrocarbon in the alkane-containing feed can be a single alkane, such as pure propane or pure butane.

In certain embodiments, the temperature sufficient to dehydrogenate the alkane ranges from 400° C. to 800° C., and all ranges and values therebetween including ranges such as 400° C. to 780° C., 420° C. to 780° C., 440° C. to 760° C., 450° C. to 750° C., 500° C. to 700° C., and 600° C. to 800° C. The reaction conditions may further include a reaction pressure of 0.2 bar to 1 bar and all ranges and values therebetween including 0.3 bar, 0.4 bar, 0.5 bar, 0.6 bar, 0.7 bar, 0.8 bar, and 0.9 bar. The reaction conditions may further include a gas hourly space velocity of 300 to 800 hr-1 and all ranges and values between the upper and lower limits of the range.

### Catalyst Preparation

### Example 1 (Comparison)

The carrier used for the catalyst preparation was prepared by dry mixing Bayerite (2524.8 g, Versal B, UOP) and chromium(III) oxide, (360.2, Sigma-Aldrich®) for 10 minutes in an Eirich mixer (EL-5 Profi Plus). An aqueous solution of nitric acid (400 ml, 25 wt. %) was added to the mixer and mixed for about 10 minutes. The obtained blend was aged at 25° C. for 1 hour and formed into cylindrical extrudates (3.5 mm diameter) using an ETP1 Bonnot lab extruder, dried at 70° C. followed by 120° C. for 12 hours, calcined at 600° C. for 2 hours in air in a muffle furnace, and cooled to room temperature for catalyst preparation.

The catalyst was prepared using this carrier. The calcined extrudates (189.51 g) were impregnated with an aqueous solution prepared by dissolving chromium nitrate nonahydrate (31.01 g), sodium nitrate (3.29 g), magnesium nitrate hexahydrate (12.72 g) and zirconium (IV) basic carbonate (1.75 g) in water (38.1 g). The impregnated extrudates were aged at 25° C. for 12 hours in a closed container. The sample was dried at 70° C. followed by 120° C. for 12 hours, calcined at 760° C. for 2 hours in air in a muffle furnace and cooled to room temperature and used for catalyst testing. The catalyst of this example had a composition of 20 wt. % Cr₂O₃, 0.60 wt. % Na₂O, 1.0 wt. % MgO, 0.7 wt. % ZrO₂ and 77.70 wt. % Al₂O₃.

### Example 2 (Invention-Catalyst with Pseudo-Boehmite)

The carrier used for the catalyst preparation was prepared by dry mixing Bayerite (2490.2 g, Versal B, UOP), pseudo-boehmite (260.7 g, Versal 250, UOP) and chromium(III) oxide, (394.8 g, Sigma-Aldrich®) for 10 minutes in an Eirich mixer (EL-5 Profi Plus). An aqueous solution of nitric acid (400 ml of 25 wt. % nitric acid mixed with 70 ml water) was added to the mixer and the mixture was mixed for about 10 minutes. The obtained blend was aged at 25° C. for 1 hour and formed into cylindrical extrudates (3.5 mm diameter) using an ETP1 Bonnot lab extruder, dried at 70° C. followed by 120° C. for 12 hours, then calcined at 600° C. for 2 hours in air in a muffle furnace, and cooled to room temperature for catalyst preparation.

The catalyst was prepared using this carrier. The calcined extrudates (189.51 g) were impregnated using a wet impregnation process with an aqueous solution prepared by dissolving chromium nitrate nonahydrate (31.01 g), sodium nitrate (3.29 g), magnesium nitrate hexahydrate (12.72 g) and zirconium (IV) basic carbonate (1.75 g) in water (38.1 g). The impregnated extrudates were aged at 25° C. for 12 hours in a closed container. The sample was dried at 70° C. followed by 120° C. for 12 hours, calcined at 760° C. for 2 hours in air in a muffle furnace and cooled to room temperature and used for catalyst testing. The catalyst of this example had a composition of 20 wt. % Cr₂O₃, 0.60 wt. % Na₂O, 1.0 wt. % MgO, 0.7 wt. % ZrO₂ and 77.70 wt. % Al₂O₃.

### Crush Strength Measurements

The catalyst extrudate crush strength was measured by ASTM D6175 method using Vinci Technologies VERSATILE CATALYST CRUSHING STRENGTH TESTER (VCS). A sample of 50 to 100 g heated at 400±15° C. for 3 h. After heating, the test sample is cooled in a desiccator to prevent the adsorption of moisture before testing. Only the number of extrudates that could be tested within a 10-min period were removed from the desiccator. The length of the extrudates were measured to the nearest tenth of a millimeter and recorded. Tweezers, forceps, or other suitable devices or procedures were used to prevent the transfer of moisture from the operator's hands to the extrudate being tested. The measured extrudates that had a length to diameter ratio greater than or equal to one, preferably extrudates with length in the range of 6 to 8 mm and diameter in the range of 2.9 to 3.3 mm, were placed between the anvils of the compression testing device. The extrudates were positioned flat against the face of the anvil and crushed radially by applying an increasing force at a uniform rate in the range of 1 to 5 lbf/s (4.4 to 22 N/s) until the extrudate crushed or collapsed. The anvils were then separated and all residue was removed with a soft brush. Care was taken to ensure that the faces of the anvils were free from particles adhering to the surface. Each extrudate was subjected to an increasing load up to breaking point and the (max load value 100 daN). The compression strength over length ratio is reported as daN/mm. The results are shown in Table 1.

The results provided in Table 1 demonstrates that the catalyst prepared according to the presently disclosed method (Example 2) is characterized by higher crush strength in comparison with the catalyst prepared by the comparative method (Example 1).

TABLE 1
Crush Strength Measurement Results

Crush strength (daN/mm)

| | Min | Average | Max | STDEV |
|---|---|---|---|---|
| Example 1 | 1.47 | 2.92 | 4.30 | 0.72 |
| Example 2 | 1.87 | 3.63 | 4.88 | 0.70 |

Number of extrudates = 30

### Catalyst Testing

The isobutane dehydrogenation activity of the catalysts of Examples 1 and 2 were tested using a fixed bed reactor. The catalyst loading and reactor details were as follows: Catalyst weight was 70 g, catalyst particle size was about 3 mm diameter, catalyst diluent quartz (ring) size was 2.2×2 mm, catalyst diluent weight ratio was 1:3, reactor inner diameter was 41 mm, reactor outer diameter was 45 mm. The catalyst extrudates (10 g of 7 batches) and inert quartz diluent (30 g of 7 batches) were loaded into reactor in layered manner. Quartz rings mentioned above were loaded above the catalyst bed. A nitrogen purge was employed between the steps of dehydrogenation, catalyst regeneration/oxidation and reduction with hydrogen. The isobutane flow in the dehydrogenation step corresponds to GHSV of 600 ml h-1 g-1. The reactor was operated at atmospheric pressure using isobutane (99.9 vol. %) diluted with nitrogen. The reaction pressure during dehydrogenation was 0.33 atmosphere of isobutane and 0.67 atmosphere of nitrogen. The reactor outlet gases were analyzed by online gas chromatograph (Agilent 6890) equipped with a flame ionization detector for hydrocarbon analysis and thermal conductivity detector for hydrogen analysis. The reactant and products flow rates were measured using a Ritter type wet gas flow meter. The reactor was operated in a cyclic mode with the following steps: 1) Catalyst oxidation/regeneration with air with a start temperature of 650° C. for 10 minutes. 2) Purge the catalyst bed with nitrogen at 650° C. for 3 minutes. 3) Reduce the catalyst with hydrogen with a start temperature of 650° C. for 3 minutes. 4) Catalyst bed cooling under nitrogen from 650° C. to 585° C. and maintaining at 585° C. for 15 minutes. 5) Isobutane dehydrogenation with a start temperature of 585° C. for 10 minutes. Reactor outlet gas composition analysis with gas chromatograph at 9th minute from the start of the isobutane feed.

Because the fresh catalyst activity changes during the initial cycles the catalysts were equilibrated under cyclic oxidation (air, 2 minutes)-purge (nitrogen, 4 minutes)-reduction (hydrogen, 2 minutes) conditions to achieve stable catalyst performance. The equilibration of the fresh catalyst was conducted at 650° C. for 100 cycles.

Steps 1 to 5 were repeated for 30 cycles and the catalyst performance results after catalyst stabilization is provided in Table 2 (21-30 cycle average). The results from Table 2 demonstrate that the catalyst prepared according to the presently disclosed method (Example 2) is characterized by similar performance in comparison with the catalyst prepared by the by the comparative method (Example 1).

TABLE 2
Catalyst Performance Results

| | Isobutane | Isobutylene | Isobutylene |
|---|---|---|---|
| Example | Conversion (mol %) | Selectivity (mol %) | Yield (mol %) |
| 1 | 40.8 | 93.3 | 38.1 |
| 2 | 38.7 | 94.8 | 36.6 |

In the context of the present invention, at least twelve embodiments are now described. Embodiment 1 is a method for making an alkane dehydrogenation catalyst. The method includes the steps of providing a chromium(III) oxide-containing alumina support containing a plurality of transition aluminas derived from a mixture containing about 60 wt. % to about 99 wt. % of crystalline aluminium trihydroxide and about 1 wt. % to about 40 wt. % of crystalline aluminium oxide-hydroxide or gelatinous aluminium hydroxide or both; contacting the chromium(III) oxide-containing alumina support with a water-soluble trivalent chromium oxide source and an alkali metal oxide source to provide an impregnated chromium(III) oxide-containing alumina support; and drying and calcining the impregnated chromium(III) oxide-containing alumina support to produce an alkane dehydrogenation catalyst containing about 60 wt. % to about 90 wt. % of alumina, about 10 wt. % to about 40 wt. % of trivalent chromium oxide, and about 0.1 wt. % to about 5 wt. % of the alkali metal oxide. Embodiment 2 is the method of embodiment 1, wherein the mixture contains from about 90 wt. % to about 99 wt. % of crystalline aluminum trihydroxide and from about 1 wt. % to about 10 wt. % of crystalline aluminum oxide-hydroxide or gelatinous aluminum hydroxide, or combinations thereof. Embodiment 3 is the method of embodiment 2, wherein the crystalline aluminium oxide-hydroxide contains boehmite. Embodiment 4 is the method of embodiment 2, wherein the gelatinous aluminium hydroxide contains one or more of amorphous aluminium hydroxide and pseudoboehmite. Embodiment 5 is the method of embodiment 1 or 2, wherein the impregnated chromium(III) oxide-containing alumina support is calcined at a temperature ranging from about 700 degrees Celsius (° C.) to 800° C. Embodiment 6 is the method of embodiment 1, wherein the crystalline aluminium trihydroxide contains one or more of bayerite and nordstrandite.

Embodiment 7 is a method for dehydrogenation of an alkane. The method includes the steps of loading a reactor with a dehydrogenation catalyst produced by: providing a chromium(III) oxide-containing alumina support containing a plurality of transition aluminas derived from a mixture containing about 60 wt. % to about 99 wt. % of crystalline aluminium trihydroxide and about 1 wt. % to about 40 wt. % of crystalline aluminium oxide-hydroxide or gelatinous aluminium hydroxide or both; contacting the chromium(III) oxide-containing alumina support with a water-soluble trivalent chromium oxide source to provide an impregnated chromium(III) oxide-containing alumina support; and drying and calcining the impregnated chromium(III) oxide-containing alumina support to produce the dehydrogenation catalyst containing about 60 wt. % to about 90 wt. % of alumina, about 10 wt. % to about 40 wt. % of trivalent chromium oxide, and about 0.1 wt. % to about 5 wt. % of the alkali metal oxide; supplying a feed containing an alkane through the reactor at a temperature sufficient to dehydrogenate the alkane and produce an olefin. Embodiment 8 is the method of embodiment 7, wherein the mixture contains from about 90 wt. % to about 99 wt. % of crystalline aluminum trihydroxide and from about 1 wt. % to about 10 wt. % of crystalline aluminum oxide-hydroxide or gelatinous aluminum hydroxide, or combinations thereof. Embodiment 9 is the method of embodiment 7, wherein the crystalline aluminium oxide-hydroxide contains boehmite. Embodiment 10 is the method of embodiment 7, wherein the gelatinous aluminium hydroxide contains one or more of amorphous aluminium hydroxide and pseudoboehmite. Embodiment 11 is the method of embodiment 7, wherein the temperature sufficient to dehydrogenate the alkane ranges from 400° C. to 800° C. Embodiment 12 is the method of embodiment 7, wherein the crystalline aluminium trihydroxide contains one or more of bayerite and nordstrandite.

When ranges are disclosed herein, ranges from any lower limit may be combined with any upper limit to recite a range not explicitly recited. Additionally, ranges from any lower limit may be combined with any other lower limit to recite a range not explicitly recited. In the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features and advantages of the disclosure will become apparent from the foregoing detailed description and examples. It should be understood, however, that the detailed description and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein.

1. A method for making an alkane dehydrogenation catalyst, the method comprising:
providing a chromium(III) oxide-containing alumina support containing a plurality of transition aluminas derived from a mixture containing about 60 weight percent (wt. %) to about 99 wt. % of crystalline aluminium trihydroxide and about 1 wt. % to about 40 wt. % of crystalline aluminium oxide-hydroxide or gelatinous aluminium hydroxide or both;
contacting the chromium(III) oxide-containing alumina support with a water-soluble trivalent chromium oxide source and an alkali metal oxide source to provide an impregnated chromium(III) oxide-containing alumina support; and
drying and calcining the impregnated chromium(III) oxide-containing alumina support to produce an alkane dehydrogenation catalyst containing about 60 wt. % to about 90 wt. % of alumina, about 10 wt. % to about 40 wt. % of trivalent chromium oxide, and about 0.1 wt. % to about 5 wt. % of the alkali metal oxide.

2. The method of claim 1, wherein the mixture contains from about 90 wt. % to about 99 wt. % of crystalline aluminum trihydroxide and from about 1 wt. % to about 10 wt. % of crystalline aluminum oxide-hydroxide or gelatinous aluminum hydroxide, or combinations thereof.

3. The method of claim 2, wherein the crystalline aluminium oxide-hydroxide contains boehmite.

4. The method of claim 2, wherein the gelatinous aluminium hydroxide contains one or more of amorphous aluminium hydroxide and pseudoboehmite.

5. The method of claim 1, wherein the impregnated chromium(III) oxide-containing alumina support is calcined at a temperature ranging from about 700 degrees Celsius (° C.) to 800° C.

6. The method of claim 1, wherein the crystalline aluminium trihydroxide contains one or more of bayerite and nordstrandite.

7. A method for dehydrogenation of an alkane, the method comprising loading a reactor with a dehydrogenation catalyst produced by:
providing a chromium(III) oxide-containing alumina support containing a plurality of transition aluminas derived from a mixture containing about 60 weight percent (wt. %) to about 99 wt. % of crystalline aluminium trihydroxide and about 1 wt. % to about 40 wt. % of crystalline aluminium oxide-hydroxide or gelatinous aluminium hydroxide or both;
contacting the chromium(III) oxide-containing alumina support with a water-soluble trivalent chromium oxide source to provide an impregnated chromium(III) oxide-containing alumina support; and
drying and calcining the impregnated chromium(III) oxide-containing alumina support to produce the dehydrogenation catalyst containing about 60 wt. % to about 90 wt. % of alumina, about 10 wt. % to about 40 wt. % of trivalent chromium oxide, and about 0.1 wt. % to about 5 wt. % of the alkali metal oxide;
supplying a feed containing an alkane through the reactor at a temperature sufficient to dehydrogenate the alkane and produce an olefin.

8. The method of claim 7, wherein the mixture contains from about 90 wt. % to about 99 wt. % of crystalline aluminum trihydroxide and from about 1 wt. % to about 10 wt. % of crystalline aluminum oxide-hydroxide or gelatinous aluminum hydroxide, or combinations thereof.

9. The method of claim 7, wherein the crystalline aluminium oxide-hydroxide contains boehmite.

10. The method of claim 7, wherein the gelatinous aluminium hydroxide contains one or more of amorphous aluminium hydroxide and pseudoboehmite.

11. The method of claim 7, wherein the temperature sufficient to dehydrogenate the alkane ranges from 400° C. to 800° C.

12. The method of claim 7, wherein the crystalline aluminium trihydroxide contains one or more of bayerite and nordstrandite.
